(19) United States
(12) Patent Application Publication
(10) Pub. No.: US 2026/0137905 A1
(43) Pub. Date: May 21, 2026

(54) POLYMER REINFORCED MEDICAL DEVICE STEERING

(71) Applicant: Boston Scientific Scimed, Inc., Maple Grove, MN (US)

(72) Inventors: Ian Major, Acton (CA); Mark Phillip Olson, St. Anthony, MN (US); Jason John Matteson, JR., Beldenville, WI (US)

(21) Appl. No.: 19/388,219

(22) Filed: Nov. 13, 2025

Related U.S. Application Data
(60) Provisional application No. 63/722,968, filed on Nov. 20, 2024.

(51) Int. Cl. A61M 25/01; A61B 18/14; A61B 18/00

(52) U.S. Cl. CPC A61M 25/0147; A61B 18/1492; A61B 2018/00351; A61M 2210/125

(57) ABSTRACT

A steerable medical device includes an elongated tubular member having a proximal portion having a proximal end, a distal portion having a distal end, and an intermediate portion extending between the proximal portion and the distal portion. The elongated tubular member includes an inner sleeve having an outer surface. First and second guide members are located adjacent one another along the outer surface of the inner sleeve. A steering wire extends through the first and second guide members such that the steering wire distal portion is in the first guide member, the steering wire proximal portion is in the second guide member, and the intermediate portion includes an exposed intermediate portion between the first guide member and the second guide member.

### CROSS REFERENCE TO RELATED APPLICATIONS

This application claims priority to U.S. Provisional Patent Application No. 63/722,968 entitled “POLYMER REINFORCED MEDICAL DEVICE STEERING,” filed November 20, 2024, which is hereby incorporated by reference in its entirety.

### TECHNICAL FIELD

The present disclosure relates to medical systems and methods for steering medical devices in a patient. More specifically, the present disclosure relates to medical devices including steering mechanisms.

### BACKGROUND

Steerable medical devices, for example steerable catheters, are currently costly to manufacture due to expensive components, labor, and overhead costs stemming from complex designs and complex assembly methods. Many steerable devices utilize a metallic pull ring welded to one or more steering wires. This type of pull ring assembly requires high tolerance, high cost, and much labor time to manufacture.

The improvements described herein improves the manufacturability of steerable medical devices by reducing components, assembly steps, and supplier risk. This is achievable by eliminating the pull ring component and providing an alternate steering mechanism focused on anchored pull wires.

### SUMMARY

Example 1 is a steerable medical device. The medical device includes an elongated tubular member having a proximal portion having a proximal end, a distal portion having a distal end, and an intermediate portion extending between the proximal portion and the distal portion. The elongated tubular member includes an inner sleeve having an outer surface. First and second guide members are located adjacent one another along the outer surface of the inner sleeve. A steering wire includes a proximal portion having a proximal end, a distal portion having a distal end, and an intermediate portion extending between the proximal portion and the distal portion. The steering wire extends through the first and second guide members such that the steering wire distal portion is in the first guide member, the steering wire proximal portion is in the second guide member, and the intermediate portion includes an exposed intermediate portion between the first guide member and the second guide member.

Example 2 is the steerable medical device of Example 1, wherein the exposed intermediate portion of the steering wire forms an approximately 180-degree curve.

Example 3 is the steerable medical device of Examples 1 or 2, wherein a distal end of the first and second guide members is proximal to the distal end of the elongated tubular member.

Example 4 is the steerable medical device of any of Examples 1 to 3, further comprising a stiff segment of material surrounding the exposed intermediate portion of the steering wire.

Example 5 is the steerable medical device of Example 4, wherein the stiff segment of material comprises a polymer.

Example 6 is the steerable medical device of any of Examples 1 to 5, further comprising a support layer.

Example 7 is the steerable medical device of Example 6, wherein the support layer surrounds a portion of the first and second guide members.

Example 8 is the steerable medical device of Example 6, wherein the first and second guide members are located between the support layer and the outer surface of the inner sleeve.

Example 9 is the steerable medical device of any of Examples 6 to 8, further comprising a layer of material encasing the support layer and the first and second guide members.

Example 10 is the steerable medical device of any of Examples 5 to 9, wherein the support layer is one or more braid, coil, mesh, slotted hypotube, or etched hypotube.

Example 11 is the steerable medical device of any of Examples 1 to 10, further comprising third and fourth guide members located adjacent one another along the outer surface of the inner sleeve, and an additional steering wire. The additional steering wire includes a proximal portion, a distal portion, and an intermediate portion extending between the proximal portion and the distal portion. The additional steering wire extends through the third and fourth guide members such that the additional steering wire distal portion is in the third guide member, the additional steering wire proximal portion is in the fourth guide member, and the additional steering wire intermediate portion includes an exposed intermediate portion between the third guide member and the fourth guide member.

Example 12 is the steerable medical device of Example 11, wherein the first and second guide members are positioned on an opposite side of the outer surface from the third and fourth guide members.

Example 13 is the steerable medical device of any of Examples 1 to 12, further comprising an actuator connected to the proximal end and the distal end of the steering wire, wherein actuation of the actuator causes the medical device to move towards a curved configuration.

Example 14 is the steerable medical device of any of Examples 1 to 14, wherein the distal portion of the elongated tubular member tapers towards the distal end.

Example 15 is the steerable medical device of any of Examples 1 to 14, wherein the elongated tubular member includes a lumen extending between the proximal end and the distal end.

Example 16 is a steerable medical device. The medical device includes an elongated tubular member having a proximal portion having a proximal end, a distal portion having a distal end, and an intermediate portion extending between the proximal portion and the distal portion. The elongated tubular member includes an inner sleeve having an outer surface. First and second guide members are located adjacent one another along the outer surface of the inner sleeve. A steering wire includes a proximal portion having a proximal end, a distal portion having a distal end, and an intermediate portion extending between the proximal portion and the distal portion. The steering wire extends through the first and second guide members such that the steering wire distal portion is in the first guide member, the steering wire proximal portion is in the second guide member, and the intermediate portion includes an exposed intermediate portion between the first guide member and the second guide member. A stiff segment of material surrounds the exposed intermediate portion of the steering wire.

Example 17 is the steerable medical device of Example 16, wherein the exposed intermediate portion of the steering wire forms an approximately 180-degree curve.

Example 18 is the steerable medical device of Example 16, wherein a distal end of the first and second guide members is proximal to the distal end of the elongated tubular member.

Example 19 is the steerable medical device of Example 16, wherein the stiff segment of material comprises a polymer.

Example 20 is the steerable medical device of Example 16, further comprising a support layer.

Example 21 is the steerable medical device of Example 20, wherein the support layer surrounds a portion of the first and second guide members.

Example 22 is the steerable medical device of Example 20, wherein the first and second guide members are located between the support layer and the outer surface of the inner sleeve.

Example 23 is the steerable medical device of Example 20, further comprising a layer of material encasing the support layer and the first and second guide members.

Example 24 is the steerable medical device of Example 20, wherein the support layer is one or more braid, coil, mesh, slotted hypotube, or etched hypotube.

Example 25 is the steerable medical device of Example 20, wherein the support layer includes a fabric or metal.

Example 26 is the steerable medical device of Example 16, further comprising third and fourth guide members located adjacent one another along the outer surface of the inner sleeve. An additional steering wire having a proximal portion, a distal portion, and an intermediate portion extending between the proximal portion and the distal portion. The additional steering wire extends through the third and fourth guide members such that the additional steering wire distal portion is in the third guide member, the additional steering wire proximal portion is in the fourth guide member, and the additional steering wire intermediate portion includes an exposed intermediate portion between the third guide member and the fourth guide member.

Example 27 is the steerable medical device of Example 26, wherein the first and second guide members are positioned on an opposite side of the outer surface from the third and fourth guide members.

Example 28 is the steerable medical device of Example 16, further comprising an actuator connected to the proximal end and the distal end of the steering wire, wherein actuation of the actuator causes the medical device to move towards a curved configuration.

Example 29 is the steerable medical device of Example 16, wherein the distal portion of the elongated tubular member tapers towards the distal end.

Example 30 is the steerable medical device of Example 16, wherein the elongated tubular member includes a lumen extending between the proximal end and the distal end.

Example 31 is a steerable medical device. The medical device includes an elongated tubular member including a proximal portion having a proximal end, a distal portion having a distal end, and an intermediate portion extending between the proximal portion and the distal portion. The elongated tubular member includes an inner sleeve having an outer surface. First and second guide members are located adjacent one another along the outer surface of the inner sleeve. A support layer surrounds a portion of the first and second guide members. A steering wire includes a proximal portion having a proximal end, a distal portion having a distal end, and an intermediate portion extending between the proximal portion and the distal portion. The steering wire extends through the first and second guide members such that the steering wire distal portion is in the first guide member, the steering wire proximal portion is in the second guide member, and the intermediate portion includes an exposed intermediate portion between the first guide member and the second guide member. A stiff segment of material surrounds the exposed intermediate portion of the steering wire.

Example 32 is the steerable medical device of Example 31, further comprising a layer of material encasing the support layer and the first and second guide members.

Example 33 is the steerable medical device of Example 31, further comprising third and fourth guide members located adjacent one another along the outer surface of the inner sleeve. An additional steering wire includes a proximal portion, a distal portion, and an intermediate portion extending between the proximal portion and the distal portion. The additional steering wire extends through the third and fourth guide members such that the additional steering wire distal portion is in the third guide member, the additional steering wire proximal portion is in the fourth guide member, and the additional steering wire intermediate portion includes an exposed intermediate portion between the third guide member and the fourth guide member.

Example 34 is a steerable medical device. The medical device includes an elongated tubular member having a proximal portion having a proximal end, a distal portion having a distal end, and an intermediate portion extending between the proximal portion and the distal portion. The elongated tubular member includes an inner sleeve having outer surface. First and second guide members are located adjacent one another along the outer surface of the inner sleeve. Third and fourth guide members are located adjacent one another along the outer surface of the inner sleeve opposite the first and second guide members. A steering wire includes a proximal portion having a proximal end, a distal portion having a distal end, and an intermediate portion extending between the proximal portion and the distal portion. The steering wire extends through the first and second guide members such that the steering wire distal portion is in the first guide member, the steering wire proximal portion is in the second guide member, and the intermediate portion includes an exposed intermediate portion between the first guide member and the second guide member. An additional steering wire includes a proximal portion, a distal portion, and an intermediate portion extending between the proximal portion and the distal portion. The additional steering wire extends through the third and fourth guide members such that the additional steering wire distal portion is in the third guide member, the additional steering wire proximal portion is in the fourth guide member, and the additional steering wire intermediate portion includes an exposed intermediate portion between the third guide member and the fourth guide member. A stiff segment of material surrounds the exposed intermediate portions of the steering wire and the additional steering wire.

Example 35 is the steerable medical device of Example 34, further comprising a support layer surrounding a portion of the first, second, third, and fourth guide members and the elongated tubular member.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIGS. 1A - 1C are schematic illustrations of a medical procedure within a patient’s heart utilizing a transseptal access system, in accordance with embodiments of the disclosure.

FIG. 2 is a perspective view of a steerable medical device, in accordance with embodiments of the disclosure.

FIGS. 3A, 3B, and 3C are cross-sectional views of the steerable medical device along line 3-3 in FIG. 2, in accordance with embodiments of the disclosure.

FIG. 4 is a perspective, partial cross-sectional view of a steerable medical device during assembly, in accordance with embodiments of the disclosure.

FIG. 5 is a perspective, partial cross-sectional view of a steerable medical device during assembly, in accordance with embodiments of the disclosure.

FIG. 6 is a perspective, partial cross-sectional view of a steerable medical device during assembly, in accordance with embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

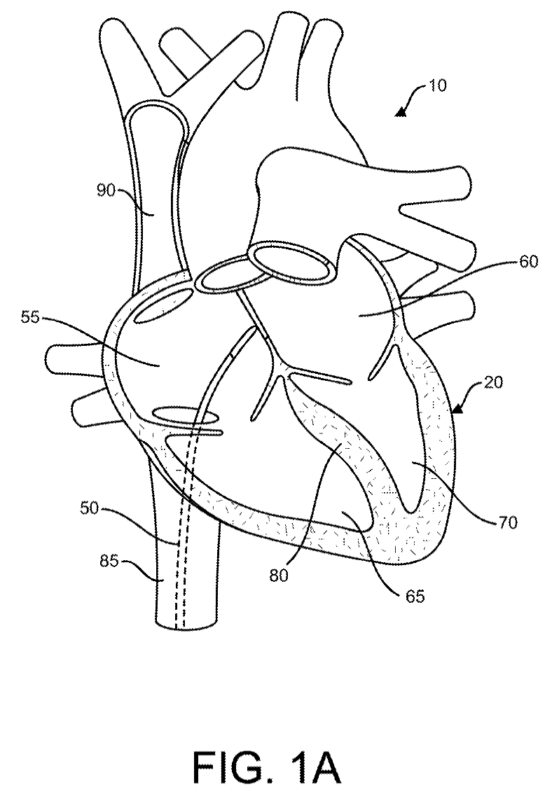

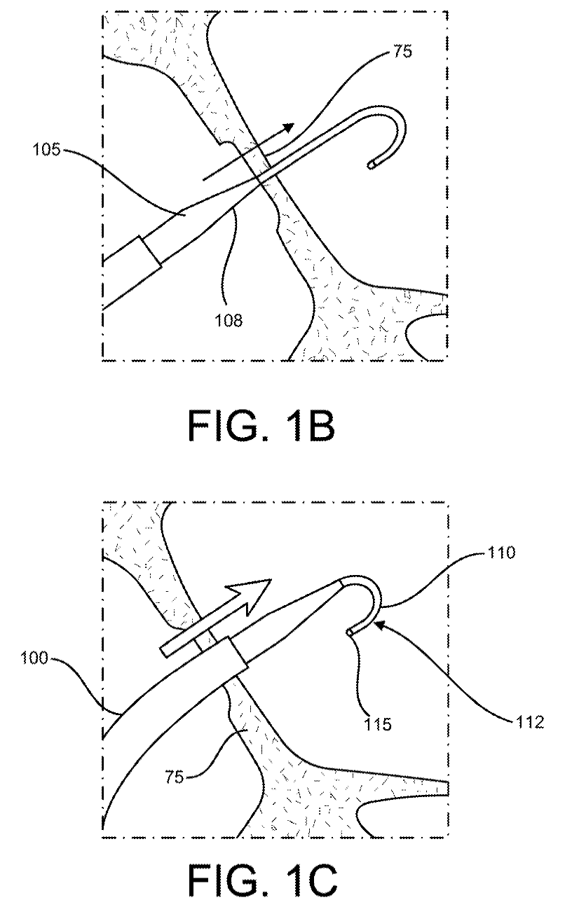

FIGS. 1A-1C are schematic illustrations of a medical procedure 10 within a patient’s heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient’s body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a radiofrequency (RF) perforation device 110, also known as a piercing device, having distal end portion 112 terminating in a tip electrode 115. As shown, in the assembled use state illustrated in FIGS. 1A-1C, the RF perforation device 110 can be disposed within the dilator 105, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100, and dilator 105 have been positioned in the superior vena cava, the guidewire is removed from the body, and the sheath 100 and the dilator 105 are retracted so that their distal ends are positioned in the right atrium 55. The RF perforation device 110 described can then be introduced into the dilator 105, and advanced toward the heart 20. In some aspects, the guidewire may itself include an RF electrode so as to function as an RF perforation device.

Subsequently, the user may position the distal end of the dilator 105 against the atrial septum 75, which can be done under imaging guidance. The RF perforation device 110 is then positioned such that electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 200 V RMS (565 V peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 s and about 5 s.

With the tip electrode 115 of the RF perforation device 110 having crossed the atrial septum 75, the dilator 105 can be advanced forward, with the tapered distal tip portion 108 operating to gradually enlarge the perforation to permit advancement of the distal end of the sheath 100 into the left atrium 60.

In some embodiments, the distal end portion 112 of the RF perforation device 110 is pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF perforation devices can be found, for example, in U.S. Patent Application Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60 and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

The transseptal access system 50 is configured to achieve a plurality of different curvatures. This is useful to allow introduction into and positioning of the system 50 at a desired location within the heart 20. For example, the various curvatures allow for achieving desired positioning of the dilator 105 and the RF perforation device 110 along a portion of the atrial septum 75.

In some aspects, the RF perforation device 110 is replaced with a mechanical piercing device such as a needle having a sharp distal tip. The needle can be configured to pierce the atrial septum 75 when the sharp distal tip is positioned on the atrial septum 75 and pressure is applied to the proximal end.

In some aspects, the dilator 105, sheath 100, or RF perforation device 110 include one or more surface electrodes. The one or more surface electrodes may be located on a distal portion of the dilator 105, sheath 100, or RF perforation device 110 for use in ablation, mapping, pacing, or sensing a parameter within a portion of the heart 20. The one or more surface electrodes may be connected to an electroanatomical mapping (EAM) system, generator, or other diagnostic system.

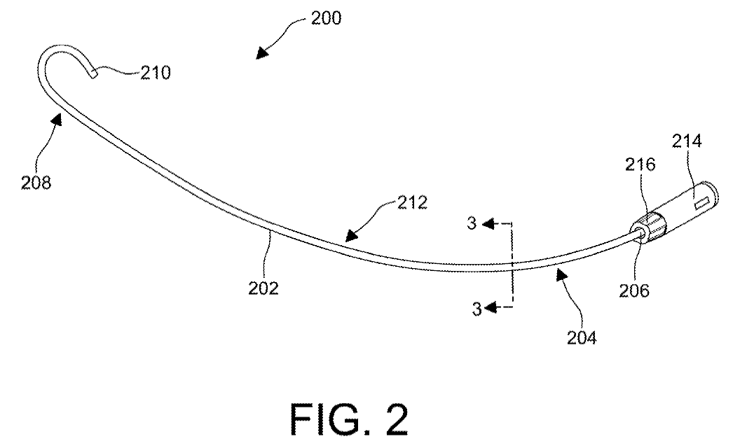

FIG. 2 is a perspective view of a steerable medical device 200, in accordance with embodiments of the disclosure. The steerable medical device 200 can take the form of a sheath, catheter, dilator, perforation device, guidewire as described above, or any other elongate medical device capable of being used within a patient. The steerable medical device 200 includes an elongated tubular member 202 having a proximal portion 204 including a proximal end 206. The elongated tubular member 202 includes a distal portion 208 having a distal end 210. An intermediate portion 212 extends between the proximal portion 204 and the distal portion 208. In some embodiments, the elongated tubular member 202 has a constant diameter from the proximal end 206 to the distal end 210. In some embodiments, the elongated tubular member 202 includes a tapered distal portion that reduces in diameter towards the distal end 201. The proximal end 206 of the elongated tubular member 202 is connected to a handle 214 having an actuator 216. Manipulation of the actuator 216 causes the distal portion 208 to bend or move towards a curved configuration to allow for steering of the device 200 through tortuous passageways within a patient.

The elongated tubular member 202 is formed of multiple layers. The elongated tubular member 202 includes an inner sleeve 218 having an outer surface 220. The inner sleeve 218 includes a lumen 222 that extends between the proximal end 206 and the distal end 210. The inner sleeve 218 is formed of a suitable polymeric material. A support member 268, a layer of material 270, and a stiff segment of material 266 also form part of the elongated tubular member 202 as discussed further below.

Located along the outer surface 220 of the inner sleeve 218 are a plurality of guide members. The guide members are elongated tubular members and are arranged along the outer surface 220 in pairs adjacent one another. Each pair of guide members is configured to receive a single steering wire as discussed below.

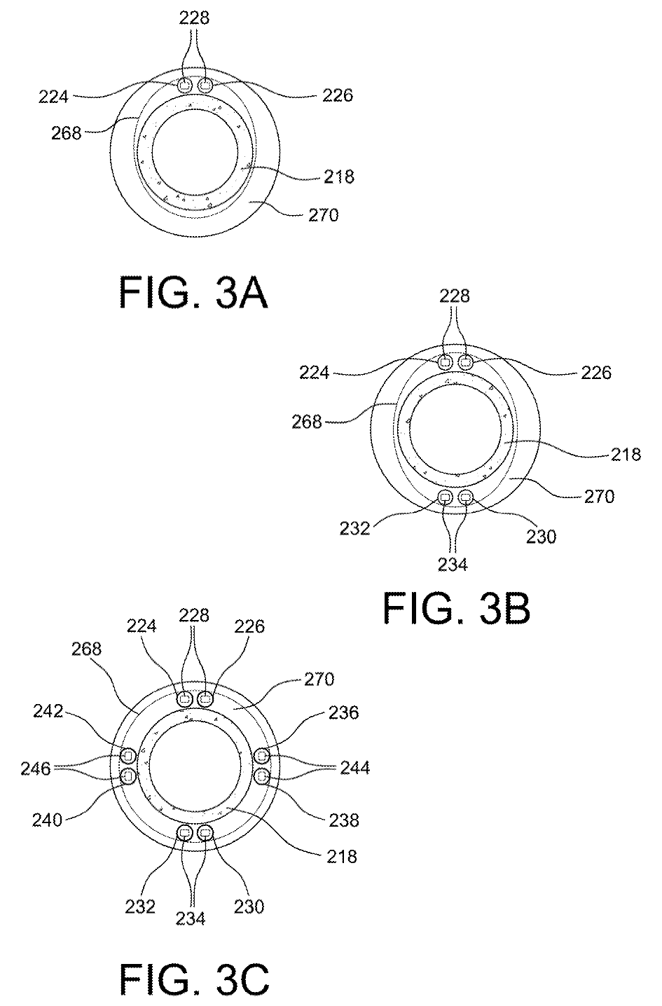

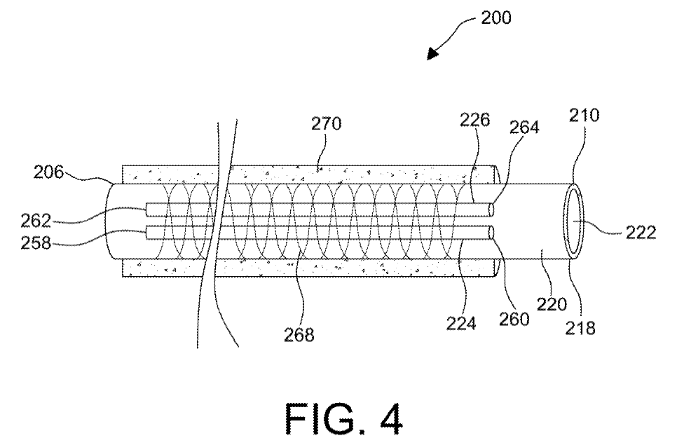

FIGS. 3A, 3B, and 3C are cross-sectional views of the steerable medical 200 device along line 3-3 in FIG. 2, showing various arrangements for pairs of guide members in accordance with embodiments of the disclosure. FIG. 3A illustrates a cross-section of a steerable medical device 200 having a single pair of guide members. The single pair of guide members includes a first guide member 224 and a second guide member 226. As illustrated in FIG. 4, the first guide member 224 and the second guide member 226 are located adjacent one another along the outer surface 220 of the inner sleeve 218. The first guide member 224 and the second guide member 226 are substantially parallel to one another along the outer surface 220. The first guide member 224 includes a proximal end 258 and a distal end 260. The second guide member 226 includes a proximal end 264 and a distal end 264. A single steering wire 228 is located within the first guide member 224 and the second guide member 226. This arrangement allows for curving of the medical device 200 in one direction as the steering wire 228 held by the single pair of guide members is manipulated by the actuator 216. As shown in FIG. 3A, and discussed further below, the first guide member 224 and the second guide member 226 are encased or embedded in a layer of material 270. The elongated tubular member 202 also includes a support layer 268 encased or embedded within the layer of material 270. The support layer 268 surrounds at least a portion the first guide member 224 and the second guide member 226.

FIG. 3B illustrates a cross-section of a steerable medical device 200 having two pairs of guide members. The first pair of guide members includes the first guide member 224 and the second guide member 226. A second pair of guide members includes a third guide member 230 and a fourth guide member 232. The first guide member 224 and the second guide member 226 are located on an opposite side of the inner sleeve as the third guide member 230 and the fourth guide member 232. Like the first pair of guide members, the third guide member 230 and the fourth guide member 232 are substantially parallel to one another along the outer surface 220. A single steering wire 234 is located within the third guide member 230 and the fourth guide member 232. This arrangement allows for curving of the medical device 200 in two directions. The steering wire 228 held by the first pair of guide members and manipulated by the actuator 216 allows for curvature of the medical device 200 in a first direction. The steering wire 234 held by the second pair of guide members and manipulated by the actuator 216 allows for curvature of the medical device 200 in a second direction that is substantially opposite the first direction. As shown in FIG. 3B, and discussed further below, the first guide member 224, the second guide member 226, the third guide member 230, and the fourth guide member 232 are encased or embedded in the layer of material 270. The elongated tubular member 202 also includes a support layer 268 encased or embedded within the layer of material 270. The support layer 268 surrounds at least a portion the first guide member 224, the second guide member 226, the third guide member 230, and the fourth guide member 232.

FIG. 3C illustrates a cross-section of a steerable medical device 200 having four pairs of guide members. The four pairs of guide members are equally spaced around the circumference of the inner sleeve 218. The first pair of guide members includes the first guide member 224 and the second guide member 226. The second pair of guide members includes the third guide member 230 and the fourth guide member 232. A third pair of guide members includes a fifth guide member 236 and a sixth guide member 238. A fourth pair of guide members includes a seventh guide member 240 and an eighth guide member 242. Like the first and second pair of guide members, the third pair of guide members and the fourth pair of guide member are substantially parallel to one another along the outer surface 220. A single steering wire 244 is located within the fifth guide member 236 and the sixth guide member 238. Additionally, a single steering wire 246 is located within the seventh guide member 240 and the eighth guide member 242. The first pair of guide members are located opposite the second pair of guide members. The third pair of guide members are located opposite the fourth pair of guide members. This arrangement allows for curving of the medical device 200 in all directions by manipulation of the steering wires 228, 234, 244, 246 associated with each of the four pairs of guide members. The steering wire 228 held by the first pair of guide members and manipulated by the actuator 216 allows for curvature in a first direction. The steering wire 234 held by the second pair of guide members and manipulated by the actuator 216 allows for curvature in a second direction that is substantially opposite the first direction. The steering wire 244 held by the third pair of guide members and manipulated by the actuator 216 allows for curvature in a third direction. The steering wire 246 held by the fourth pair of guide members and manipulated by the actuator 216 allows for curvature in a fourth direction that is substantially opposite the third direction. Manipulation of more than one steering wire together allows for curving in directions between the first, second, third, and fourth directions. As shown in FIG. 3C, and discussed further below, the first guide member 224, the second guide member 226, the third guide member 230, the fourth guide member 232, the fifth guide member 236, the sixth guide member 238, the seventh guide member 240, and the eighth guide member 242 are encased or embedded in the layer of material 270. The elongated tubular member 202 also includes a support layer 268 encased or embedded within the layer of material 270. The support layer 268 surrounds at least a portion of the first guide member 224, the second guide member 226, the third guide member 230, the fourth guide member 232, the fifth guide member 236, the sixth guide member 238, the seventh guide member 240, and the eighth guide member 242.

FIG. 4 illustrates how a single steering wire is positioned in a pair of guide members. While FIG. 4 shows only the first pair of guide members, first guide member 224 and second guide member 226, the arrangement would be the same for any of the pairs of guide members and an associated steering wire. Steering wire 228 is a continuous filament. The steering wire 228 can be formed of a single strand of material, or of a plurality of strands of material. For instance, the steering wire 228 can take the form of a cable or braided wired. The steering wire 228 includes a proximal portion 248 having a proximal end 250, a distal portion 252 having a distal end 254, and an intermediate portion 256 extending between the proximal portion 248 and the distal portion 252. The steering wire 228 can have a cross-section in the form of a circle, oval, rectangle, square, or polygon to achieve desired mechanical properties for introduction into and translation along the pair of guide members. In some embodiments, the steering wire 228 has a constant cross-sectional area along the entire length thereof. In other embodiments, the steering wire 228 has a reduced cross-sectional area in the intermediate portion 256 to aid in bending of the steering wire 228.

The steering wire 228 is continuous and arranged to extend through both the first guide member 224 and the second guide member 226. The steering wire 228 forms a generally u-shaped arrangement with free ends proximal of the first guide member 224 and the second guide member 226. The free ends, including the distal end 254 and the proximal end 250, are attached to the actuator 216 such that the actuator 216 simultaneously acts on both the distal end 254 and the proximal end 250 to cause bending or curving of the medical device 200.

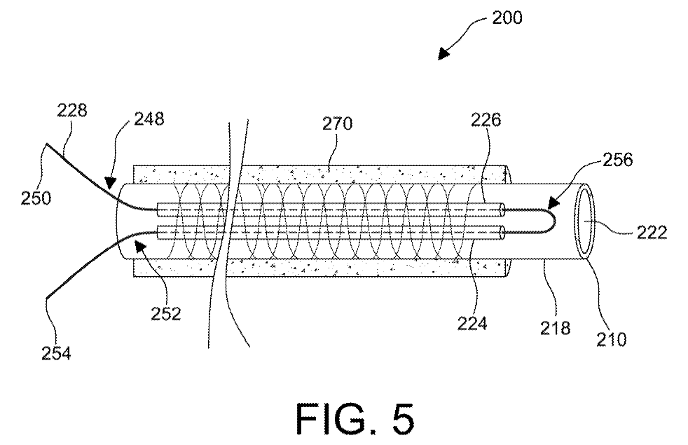

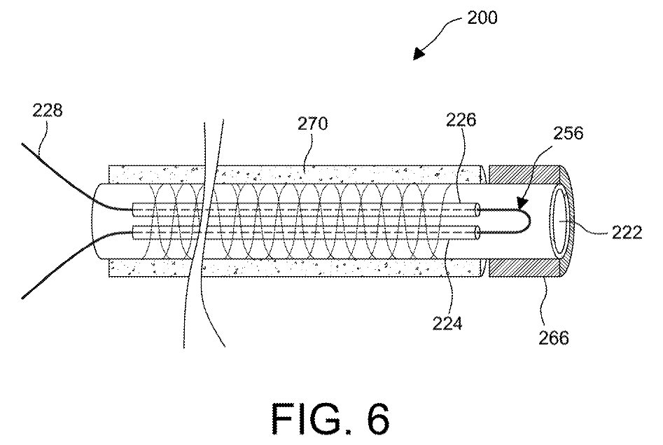

As illustrated in FIG. 5, the steering wire 228 is arranged such that part of the distal portion 252 is positioned in the first guide member 224, part of the proximal portion 248 is positioned in the second guide member 226, and part of the intermediate portion 256 is exposed between the first guide member 224 and the second guide member 226. The exposed part of the intermediate portion 256 forms an approximately 180-degree curve. The exposed part of the intermediate portion 256 is secured in place using a stiff segment of material 266 as illustrated in FIG. 6. The stiff segment of material 266 anchors the steering wire 228 such that tension applied to the distal end 254 and the proximal end 250 allows for bending or curving of the distal portion 208 of the medical device 200.

FIG. 6 is a perspective, partial cross-section view of the steerable medical device 200. As illustrated in FIG. 6, the material 266 and material 270 are cut away to expose the underlying guides 224 and 226 and the intermediate portion 256 of the steering wire 228. As shown, the stiff segment of material 266 surrounds and encases the exposed intermediate portion 256 of the steering wire 228 such that the exposed intermediate portion 256 is embedded in the stiff segment of material 266. In some embodiments, the stiff segment of material 266 also surrounds the distal end 260 of the first guide member 224 and the distal end 264 of the second guide member 226. In some embodiments, the stiff segment of material 266 flows into the first guide member 224 and the second guide member 226. In some embodiments, the stiff segment of material 266 comprises a polymer or polymeric material.

In some embodiments, the stiff segment of material 266 surrounds the entire circumference of the inner sleeve 218. In some embodiments, the exposed intermediate portion 256 is anchored by the stiff segment of material 266 over only a portion of the circumference of the inner sleeve 218. For example, the stiff segment of material 266 is positioned only at a location corresponding to the exposed intermediate portion 256. In other embodiments, the exposed intermediate portion 256 is anchored by a mechanical device such as a pin, shoulder, or fastener in lieu of or in addition to the stiff segment of material 266.

The first guide member 224 and the second guide member 226 do not extend along the entire medical device. The distal ends 260, 264 of the first and second guide members 224, 226 are proximal to the distal end 210 of the elongated tubular member 202. This allows for space at the proximal portion 204 for the steering wire 228 to join with the actuator 216 and space at the distal potion 208 for the steering wire 228 to form the approximately 180-degree curve at the exposed intermediate portion 256.

The support layer 268 surrounds the guide members. As shown in FIGS. 3A-3C, the guide members 224, 226, 230, 232, 236, 238, 240, 242 are located between the support layer 268 and the outer surface 220 of the inner sleeve 218. In various embodiments, the support layer 268 partially encases the guide members 224, 226, 230, 232, 236, 238, 240, 242 to ensure that the guide members 224, 226, 230, 232, 236, 238, 240, 242 remain substantially parallel to a longitudinal axis of the medical device 200. This allows any steering wire located within a pair of guide members to move without binding.

In some embodiments, the support layer 268 surrounds only portion of the guide members 224, 226, 230, 232, 236, 238, 240, 242. For example, the support layer 268 may include a multifilar braid or coil, with at least one filar positioned beneath the guide members 224, 226, 230, 232, 236, 238, 240, 242 and at least one filar positioned over the guide members 224, 226, 230, 232, 236, 238, 240, 242. In some embodiments, the support layer 268 surrounds the entirety of the guide members 224, 226, 230, 232, 236, 238, 240, 242. The support layer 268 includes a material that can improve torque transmission, increase rigidity, or otherwise improve handling of the medical device 200. In some embodiments, the support layer 268 is formed of a metal. In some embodiments, the support layer 268 is formed of a fabric. In various embodiments, the support layer 268 takes the form or one or more of a braid, coil, mesh, slotted hypotube, or etched hypotube.

The layer of material 270 encases the support layer 268 and any guide members 224, 226, 230, 232, 236, 238, 240, 242. The layer of material 270 can include any polymer or polymeric material and can flow in spaces between the support layer 268 and the guide members 224, 226, 230, 232, 236, 238, 240, 242. The layer of material 270 provides a smooth outer surface for the medical device 200. In some embodiments, the layer of material 270 is selected to include one or more materials that increase lubricity of the medical device 200. In some embodiments, a coating (not shown) is applied over the layer of material 270 to increase lubricity of the medical device. In some embodiments, the layer of material 270 and the stiff segment of material 266 are formed of the same polymer or polymeric material. In some embodiments, the layer of material 270 and the stiff segment of material 266 are formed of different polymers or polymeric materials.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A steerable medical device, the medical device comprising:
an elongated tubular member including a proximal portion having a proximal end, a distal portion having a distal end, and an intermediate portion extending between the proximal portion and the distal portion, the elongated tubular member including an inner sleeve having an outer surface;
first and second guide members located adjacent one another along the outer surface of the inner sleeve;
a steering wire including a proximal portion having a proximal end, a distal portion having a distal end, and an intermediate portion extending between the proximal portion and the distal portion, the steering wire extending through the first and second guide members such that the steering wire distal portion is in the first guide member, the steering wire proximal portion is in the second guide member, and the intermediate portion includes an exposed intermediate portion between the first guide member and the second guide member; and
a stiff segment of material surrounding the exposed intermediate portion of the steering wire.

2. The steerable medical device of claim 1, wherein the exposed intermediate portion of the steering wire forms an approximately 180-degree curve.

3. The steerable medical device of claim 1, wherein a distal end of the first and second guide members is proximal to the distal end of the elongated tubular member.

4. The steerable medical device of claim 1, wherein the stiff segment of material comprises a polymer.

5. The steerable medical device of claim 1, further comprising a support layer.

6. The steerable medical device of claim 5, wherein the support layer surrounds a portion of the first and second guide members.

7. The steerable medical device of claim 5, wherein the first and second guide members are located between the support layer and the outer surface of the inner sleeve.

8. The steerable medical device of claim 5, further comprising a layer of material encasing the support layer and the first and second guide members.

9. The steerable medical device of claim 5, wherein the support layer is one or more braid, coil, mesh, slotted hypotube, or etched hypotube.

10. The steerable medical device of claim 5, wherein the support layer includes a fabric or metal.

11. The steerable medical device of claim 1, further comprising:
third and fourth guide members located adjacent one another along the outer surface of the inner sleeve; and
an additional steering wire having a proximal portion, a distal portion, and an intermediate portion extending between the proximal portion and the distal portion, the additional steering wire extending through the third and fourth guide members such that the additional steering wire distal portion is in the third guide member, the additional steering wire proximal portion is in the fourth guide member, and the additional steering wire intermediate portion includes an exposed intermediate portion between the third guide member and the fourth guide member.

12. The steerable medical device of claim 11, wherein the first and second guide members are positioned on an opposite side of the outer surface from the third and fourth guide members.

13. The steerable medical device of claim 1, further comprising an actuator connected to the proximal end and the distal end of the steering wire, wherein actuation of the actuator causes the medical device to move towards a curved configuration.

14. The steerable medical device of claim 1, wherein the distal portion of the elongated tubular member tapers towards the distal end.

15. The steerable medical device of claim 1, wherein the elongated tubular member includes a lumen extending between the proximal end and the distal end.

16. A steerable medical device, the medical device comprising:
an elongated tubular member including a proximal portion having a proximal end, a distal portion having a distal end, and an intermediate portion extending between the proximal portion and the distal portion, the elongated tubular member including an inner sleeve having an outer surface;
first and second guide members located adjacent one another along the outer surface of the inner sleeve;
a support layer surrounding a portion of the first and second guide members;
a steering wire including a proximal portion having a proximal end, a distal portion having a distal end, and an intermediate portion extending between the proximal portion and the distal portion, the steering wire extending through the first and second guide members such that the steering wire distal portion is in the first guide member, the steering wire proximal portion is in the second guide member, and the intermediate portion includes an exposed intermediate portion between the first guide member and the second guide member; and
a stiff segment of material surrounding the exposed intermediate portion of the steering wire.

17. The steerable medical device of claim 16, further comprising a layer of material encasing the support layer and the first and second guide members.

18. The steerable medical device of claim 16, further comprising:
third and fourth guide members located adjacent one another along the outer surface of the inner sleeve; and
an additional steering wire having a proximal portion, a distal portion, and an intermediate portion extending between the proximal portion and the distal portion, the additional steering wire extending through the third and fourth guide members such that the additional steering wire distal portion is in the third guide member, the additional steering wire proximal portion is in the fourth guide member, and the additional steering wire intermediate portion includes an exposed intermediate portion between the third guide member and the fourth guide member.

19. A steerable medical device, the medical device comprising:
an elongated tubular member including a proximal portion having a proximal end, a distal portion having a distal end, and an intermediate portion extending between the proximal portion and the distal portion, the elongated tubular member including an inner sleeve having an outer surface;
first and second guide members located adjacent one another along the outer surface of the inner sleeve;
third and fourth guide members located adjacent one another along the outer surface of the inner sleeve opposite the first and second guide members;
a steering wire including a proximal portion having a proximal end, a distal portion having a distal end, and an intermediate portion extending between the proximal portion and the distal portion, the steering wire extending through the first and second guide members such that the steering wire distal portion is in the first guide member, the steering wire proximal portion is in the second guide member, and the intermediate portion includes an exposed intermediate portion between the first guide member and the second guide member;
an additional steering wire having a proximal portion, a distal portion, and an intermediate portion extending between the proximal portion and the distal portion, the additional steering wire extending through the third and fourth guide members such that the additional steering wire distal portion is in the third guide member, the additional steering wire proximal portion is in the fourth guide member, and the additional steering wire intermediate portion includes an exposed intermediate portion between the third guide member and the fourth guide member; and
a stiff segment of material surrounding the exposed intermediate portions of the steering wire and the additional steering wire.

20. The steerable medical device of claim 19, further comprising a support layer surrounding a portion of the first, second, third, and fourth guide members and the inner sleeve.
